(19) United States
(12) Patent Application Publication
(10) Pub. No.: US 2005/0197569 A1
(43) Pub. Date: Sep. 8, 2005

(54) Methods, systems, and apparatuses for providing patient-mounted surgical navigational sensors

(76) Inventor: Daniel McCombs, Memphis, TN (US)

Correspondence Address: CHIEF PATENT COUNSEL;SMITH & NEPHEW, INC., 1450 BROOKS ROAD, MEMPHIS, TN, 38116, US

(21) Appl. No.: 11/041,691

(22) Filed: Jan. 24, 2005

Related U.S. Application Data
(60) Provisional application No. 60/538,448, filed on Jan. 22, 2004.

(51) Int. Cl. A61B 5/05

(52) U.S. Cl. 600/426

(57) ABSTRACT

Methods and apparatuses for providing a patient-mounted navigational sensor for use in computer-aided surgery are disclosed. A navigational sensor according to embodiments of the invention includes at least two optical tracking cameras for sensing surgical references and a mount adapted to be attached to the bone of a patient. Because the sensor is mounted to the bone rather than to external apparatus, and is thus closer to the surgical references that it is tracking, it is less likely that line of sight issues due to an acute angle between the plane of the surgical reference and the sensor or due to medical personnel obstructing the path of the reference's signal. Because the navigational sensor is much closer to the surgical references being tracked than in a typical computer-aid surgery scenario, the required camera separation is greatly reduced. Other advantages also accrue from such sensor positioning, as related more fully in this document.

### RELATED APPLICATION

The present application claims priority to U.S. Provisional Ser. No. 60/538,448, entitled “Patient Mounted Navigational Camera System,” filed on Jan. 22, 2004, the contents of which are incorporated herein by reference.

### FIELD OF THE INVENTION

The invention relates to computer-aided surgery, and more particularly relates to methods, systems, and apparatuses for providing a patient-mounted navigational sensor for use in computer-aided surgery.

### BACKGROUND

Many surgical procedures require a wide array of instrumentation and other surgical items. Necessary items may include, but are not limited to: sleeves to serve as entry tools, working channels, drill guides and tissue protectors; scalpels; entry awls; guide pins; reamers; reducers; distractors; guide rods; endoscopes; arthroscopes; saws; drills; screwdrivers; awls; taps; osteotomes and wrenches. In many surgical procedures, including orthopedic procedures, it may be desirable to associate some or all of these items with a guide and/or handle incorporating a surgical reference, allowing the instrument to be used with a computer-aided surgical navigation system.

Several manufacturers currently produce computer-aided surgical navigation systems. The TREON™ and ION™ systems with FLUORONAV™ software manufactured by Medtronic Surgical Navigation Technologies, Inc. are examples of such systems. The BrainLAB VECTORVISION™ system is another example of such a surgical navigation system. Systems and processes for accomplishing computer-aided surgery are also disclosed in U.S. Ser. No. 10/084,012, filed Feb. 27, 2002 and entitled “Total Knee Arthroplasty Systems and Processes”; U.S. Ser. No. 10/084,278, filed Feb. 27, 2002 and entitled “Surgical Navigation Systems and Processes for Unicompartmental Knee Arthroplasty”; U.S. Ser. No. 10/084,291, filed Feb. 27, 2002 and entitled “Surgical Navigation Systems and Processes for High Tibial Osteotomy”; International Application No. US02/05955, filed Feb. 27, 2002 and entitled “Total Knee Arthroplasty Systems and Processes”; International Application No. US02/05956, filed Feb. 27, 2002 and entitled “Surgical Navigation Systems and Processes for Unicompartmental Knee Arthroplasty”; International Application No. US02/05783 entitled “Surgical Navigation Systems and Processes for High Tibial Osteotomy”; U.S. Ser. No. 10/364,859, filed Feb. 11, 2003 and entitled “Image Guided Fracture Reduction,” which claims priority to U.S. Ser. No. 60/355,886, filed Feb. 11, 2002 and entitled “Image Guided Fracture Reduction”; U.S. Ser. No. 60/271,818, filed Feb. 27, 2001 and entitled “Image Guided System for Arthroplasty”; and U.S. Ser. No. 10/229,372, filed Aug. 27, 2002 and entitled “Image Computer Assisted Knee Arthroplasty”, the entire contents of each of which are incorporated herein by reference as are all documents incorporated by reference therein.

These systems and processes use position and/or orientation tracking sensors such as infrared sensors acting stereoscopically or other sensors acting in conjunction with surgical references to track positions of body parts, surgery-related items such as implements, instrumentation, trial prosthetics, prosthetic components, and virtual constructs or references such as rotational axes which have been calculated and stored based on designation of bone landmarks. Sensors, such as cameras, detectors, and other similar devices, are typically mounted overhead with respect to body parts and surgery-related items to receive, sense, or otherwise detect positions and/or orientations of the body parts and surgery-related items. Processing capability such as any desired form of computer functionality, whether standalone, networked, or otherwise, takes into account the position and orientation information as to various items in the position sensing field (which may correspond generally or specifically to all or portions or more than all of the surgical field) based on sensed position and orientation of their associated surgical references, or based on stored position and/or orientation information. The processing functionality correlates this position and orientation information for each object with stored information, such as a computerized fluoroscopic imaged file, a wire frame data file for rendering a representation of an instrument component, trial prosthesis or actual prosthesis, or a computer generated file relating to a reference, mechanical, rotational or other axis or other virtual construct or reference. The processing functionality then displays position and orientation of these objects on a rendering functionality, such as a screen, monitor, or otherwise, in combination with image information or navigational information such as a reference, mechanical, rotational or other axis or other virtual construct or reference. Thus, these systems or processes, by sensing the position of surgical references, can display or otherwise output useful data relating to predicted or actual position and orientation of surgical instruments, body parts, surgically related items, implants, and virtual constructs for use in navigation, assessment, and otherwise performing surgery or other operations.

Some of the surgical references used in these systems may emit or reflect infrared light that is then detected by an infrared camera. The references may be sensed actively or passively by infrared, visual, sound, magnetic, electromagnetic, x-ray or any other desired technique. An active reference emits energy, and a passive reference merely reflects energy. Some surgical references may have markers or fiducials that are traced by an infrared sensor to determine the position and orientation of the reference and thus the position and orientation of the associated instrument, item, implant component or other object to which the reference is attached.

In addition to surgical references with fixed fiducials, modular fiducials, which may be positioned independent of each other, may be used to reference points in the coordinate system. Modular fiducials may include reflective elements which may be tracked by two, sometimes more, sensors whose output may be processed in concert by associated processing functionality to geometrically calculate the position and orientation of the item to which the modular fiducial is attached. Like fixed fiducial surgical references, modular fiducials and the sensors need not be confined to the infrared spectrum—any electromagnetic, electrostatic, light, sound, radio frequency or other desired technique may be used. Similarly, modular fiducials may “actively” transmit reference information to a tracking system, as opposed to “passively” reflecting infrared or other forms of energy.

Surgical references useable with the above-identified navigation systems may be secured to any desired structure, including the above-mentioned surgical instruments and other items. The surgical references may be secured directly to the instrument or item to be referenced. However, in many instances it will not be practical or desirable to secure the surgical references to the instrument or other item. Rather, in many circumstances it will be preferred to secure the surgical references to a handle and/or a guide adapted to receive the instrument or other item. For example, drill bits and other rotating instruments cannot be tracked by securing the surgical reference directly to the rotating instrument because the reference would rotate along with the instrument. Rather, a preferred method for tracking a rotating instrument is to associate the surgical reference with the instrument or item's guide or handle.

Various arrangements and combinations of fiducials or markers, such as navigational arrays, and sensors have been implemented for use with computer-aided surgical navigation systems. Use of such navigational arrays and sensors can be affected by “line of sight” problems. That is, when the angle between the plane of the array and the sensor becomes acute, a marker may be obscured by other markers that are coplanar with it, resulting in limited visibility of the array. Similarly, because sensors are generally fixed in the operating room in an area that allows all the surgical references to be in the sensor's field of view, such as the ceiling, the transmission path of the references' signals may be obstructed by medical personnel. When all of the markers in the array cannot be seen in an image, locating the exact position of the marker relative to a patient's body can be difficult. When line of sight problems occur during a computer-aided surgical procedure, the position of the surgical instrument associated with the navigational array or the position of the navigational array itself must be realigned or repositioned, increasing the time and effort associated with the surgical procedure.

### SUMMARY

Various aspects and embodiments of the invention include computer-aided surgical navigation systems with patient-mounted navigational sensors. Such surgical navigation systems can among other things reduce the likelihood of “line of sight” problems common in computer-aided surgery.

The computer-aided surgical navigation systems of the invention can include the following:
(a) a computer program adapted to generate reference information regarding position and orientation of a patient's body part;
(b) a sensor mounted to a patient's body part, the sensor adapted to track the position of at least one surgical reference;
(c) at least one surgical reference capable of being tracked by the sensor;
(d) the computer program adapted to receive information from the sensor in order to track a position and orientation of the at least one surgical reference with respect to the body part; and
(e) a monitor adapted to receive information from the computer in order to display at least some of the reference information relating to at least one body part and the at least one surgical reference.

Other embodiments of the invention can include an apparatus such as a position sensor that may be mounted to the body of a patient. The position sensor can include at least two sensors for sensing surgical references using at least one of the following: infrared, sound, visual, magnetic, electromagnetic and x-ray; and a mount adapted to be associated with the bone of a patient. In one particular embodiment of the invention, the sensor is an optical tracking camera. In yet another embodiment of the invention, the sensor is an optical tracking camera mounted to a patient's bone such as a femur.

Still other embodiments of the invention include a method for performing computer-assisted surgery using a patient-mounted navigational sensor. The methods can include the following:
(a) mounting a navigational sensor to a body part of a patient, wherein the navigational sensor comprises:
a sensor for sensing at least one surgical reference; and
a mount adapted to be associated with the bone of a patient;
(b) mounting at least one surgical reference adjacent to an object;
(c) sensing the at least one surgical reference with the navigational sensor; and
(d) determining at least one position associated with the object based in part on at least the sensing of the at least one surgical reference.

In at least one embodiment of the invention, the sensor can be an optical tracking camera. In another embodiment of the invention, the sensor may include at least two optical tracking cameras.

### BRIEF DESCRIPTION OF THE DRAWINGS

FIG. 1 is a schematic view of a particular system embodiment for a patient-mounted navigational sensor according to embodiments of the present invention.

FIG. 2 illustrates a flowchart of a method of use for a patient-mounted navigational sensor according to an embodiment of the present invention.

FIG. 3 illustrates a flowchart of a method of use for a computer-aided surgical navigation system with a patient-mounted navigational sensor according to an embodiment of the present invention.

### DETAILED DESCRIPTION

This invention will now be described more fully with reference to the drawings, showing preferred embodiments of the invention. However, this invention can be embodied in many different forms and should not be construed as limited to the embodiments set forth.

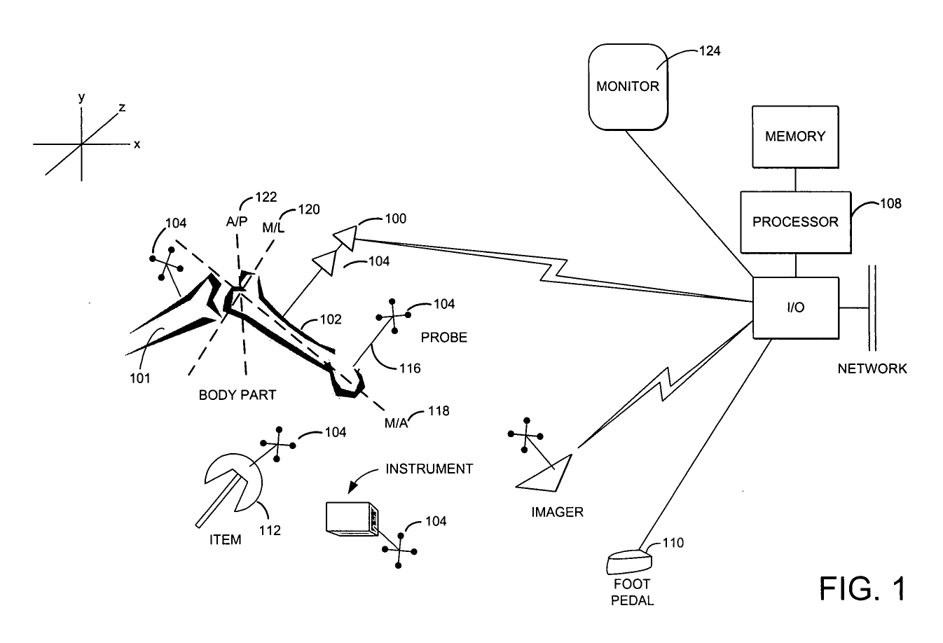

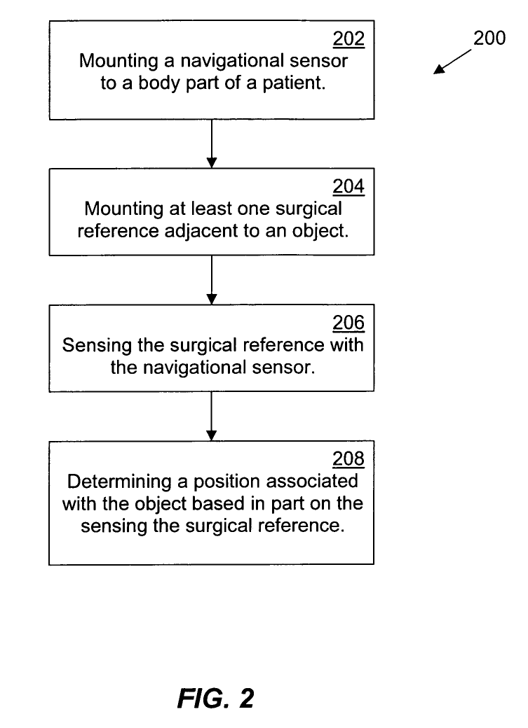

FIG. 1 is a schematic view showing an environment for using a computer-aided surgical navigation system with a patient-mounted navigational sensor according to the present invention in a surgery on a knee, in this case a knee arthroplasty. The embodiment of the computer-aided surgical navigation system shown in FIG. 1 includes a patient-mounted navigational sensor 100. A patient-mounted navigational sensor 100 according to the present invention can track particular locations associated with various body parts, such as tibia 101 and femur 102, to which surgical references 104 may be implanted, attached, or otherwise associated physically, virtually, or otherwise. The patient-mounted navigational sensor 100 may be any sort of sensor functionality for sensing the position and orientation of surgical references 104. In one embodiment, patient-mounted navigational sensor 100 can be a pair of optical tracking cameras or infrared sensors 105, 107 disposed apart from each other, and whose output can be processed in concert to provide position and orientation information regarding one or more surgical references, such as the navigational arrays 204 shown in FIG. 2. When two or more optical tracking cameras or sensors are used, the cameras or sensors can collectively provide relatively close in, and multiple viewing positions of the surgical references.

The patient-mounted navigational sensor 100 may be used to sense the position and orientation of surgical references 104 and therefore items with which they are associated. A surgical reference can include fiducial markers, such as marker elements, capable of being sensed by a navigational sensor in a computer-aided surgical navigation system. The patient-mounted navigational sensor 100 may sense active or passive signals from the surgical references 104. The signals may be electrical, magnetic, electromagnetic, sound, physical, radio frequency, optical or visual, or other active or passive technique. For example in one embodiment, the navigational sensor 100 can visually detect the presence of a passive-type surgical reference. In an example of another embodiment, the navigational sensor can receive an active signal provided by an active-type surgical reference. In the example shown in FIG. 1, the computer-aided surgical navigation system uses a patient-mounted navigational sensor 100 to sense surgical references 104. The surgical navigation system can store, process and/or output data relating to position and orientation of surgical references 104 and thus, items or body parts, such as 101 and 102 to which they are attached or associated.

As shown in FIG. 1, the patient-mounted navigational sensor 100 can be attached directly to the patient. For example, the patient-mounted navigational sensor 100 may be mounted to a body part of a patient such as the patient's femur 102. Attaching the navigational sensor 100 directly to the patient can greatly reduce “line of sight” problems experienced by conventional systems and processes. The patient-mounted navigational sensor 100 can be attached to bone or tissue anatomy in the same way that a surgical reference 104 is attached to the bone or tissue anatomy. As mentioned above, the patient-mounted navigational sensor 100 may be a two or multiple camera optical navigation system. Because the patient-mounted navigational sensor 100 is much closer to the surgical references 104 being tracked than in conventional computer-aid surgery processes and systems, the separation between any associated computer-aided surgical cameras can be greatly reduced.

In the embodiment shown in FIG. 1, computing functionality 108 such as one or more computer programs can include processing functionality, memory functionality, input/output functionality whether on a standalone or distributed basis, via any desired standard, architecture, interface and/or network topology. In one embodiment, computing functionality 108 can be connected to a monitor 114 on which graphics and data may be presented to a surgeon during surgery. The monitor 114 preferably has a tactile interface so that the surgeon may point and click on monitor 114 for tactile screen input in addition to or instead of, if desired, keyboard and mouse conventional interfaces. Additionally, a foot pedal 110 or other convenient interface may be coupled to functionality 108 as can any other wireless or wireline interface to allow the surgeon, nurse or other user to control or direct functionality 108 in order to, among other things, capture position/orientation information when certain components are oriented or aligned properly. Items 112 such as trial components, instrumentation components may be tracked in position and orientation relative to body parts 101 and 102 using one or more surgical references 104.

Computing functionality 108 can process, store and output on monitor 114 various forms of data that correspond in whole or part to body parts 200 and 202 and other components for item 112. For example, body parts 101 and 102 can be shown in cross-section or at least various internal aspects of them such as bone canals and surface structure can be shown using fluoroscopic images. These images can be obtained using a C-arm attached to a surgical reference 104. The body parts, for example, tibia 101 and femur 102, can also have surgical references 104 attached. When fluoroscopy images are obtained using the C-arm with a surgical reference 104, a patient-mounted navigational sensor 100 “sees” and tracks the position of the fluoroscopy head as well as the positions and orientations of the tibia 101 and femur 102. The computer stores the fluoroscopic images with this position/orientation information, thus correlating position and orientation of the fluoroscopic image relative to the relevant body part or parts. Thus, when the tibia 101 and corresponding surgical reference 104 move, the computer automatically and correspondingly senses the new position of tibia 200 in space and can correspondingly move implements, instruments, references, trials and/or implants on the monitor 114 relative to the image of tibia 101. Similarly, the image of the body part can be moved, both the body part and such items may be moved, or the on screen image otherwise presented to suit the preferences of the surgeon or others and carry out the imaging that is desired. Similarly, when an item 112, such as a stylus, cutting block, reamer, drill, saw, extramedullary rod, intramedullar rod, or any other type of item or instrument, that is being tracked moves, its image moves on monitor 114 so that the monitor 114 shows the item 112 in proper position and orientation on monitor 114 relative to the femur 102. The item 112 can thus appear on the monitor 114 in proper or improper alignment with respect to the mechanical axis and other features of the femur 102, as if the surgeon were able to see into the body in order to navigate and position item 112 properly.

The computer functionality 108 can also store data relating to configuration, size and other properties of items 112 such as joint replacement prostheses, implements, instrumentation, trial components, implant components and other items used in surgery. When those are introduced into the field of position/orientation sensor 100, computer functionality 108 can generate and display overlain or in combination with the fluoroscopic images of the body parts 101 and 102, computer generated images of joint replacement prostheses, implements, instrumentation components, trial components, implant components and other items 112 for navigation, positioning, assessment and other uses.

Instead of or in combination with fluoroscopic, MRI or other actual images of body parts, computer functionality 108 may store and output navigational or virtual construct data based on the sensed position and orientation of items in the surgical field, such as surgical instruments or position and orientation of body parts. For example, monitor 114 can output a resection plane, mechanical axis, anterior/posterior reference plane, medial/lateral reference plane, rotational axis or any other navigational reference or information that may be useful or desired to conduct surgery. In the case of the reference plane, for example, monitor 114 can output a resection plane that corresponds to the resection plane defined by a cutting guide whose position and orientation is being tracked by sensors 100. In other embodiments, monitor 114 can output a cutting track based on the sensed position and orientation of a reamer. Other virtual constructs can also be output on monitor 114, and can be displayed with or without the relevant surgical instrument, based on the sensed position and orientation of any surgical instrument or other item in the surgical field to assist the surgeon or other user to plan some or all of the stages of the surgical procedure.

In some embodiments of the present invention, computer functionality can output on monitor 114 the projected position and orientation of an implant component or components based on the sensed position and orientation of one or more surgical instruments associated with one or more surgical references 104. For example, the system may track the position and orientation of a cutting block as it is navigated with respect to a portion of a body part that will be resected. Computer functionality 108 may calculate and output on monitor 114 the projected placement of the implant in the body part based on the sensed position and orientation of the cutting block, in combination with, for example, the mechanical axis of the femur and/or the leg, together with axes showing the anterior/posterior and medial/lateral planes. No fluoroscopic, MRI or other actual image of the body part is displayed in some embodiments, since some hold that such imaging is unnecessary and counterproductive in the context of computer aided surgery if relevant axis and/or other navigational information is displayed. If the surgeon or other user is dissatisfied with the projected placement of the implant, the surgeon may then reposition the cutting block to evaluate the effect on projected implant position and orientation.

Additionally, computer functionality 108 can track any point in the position/orientation sensor 100 field such as by using a designator or a probe 116. The probe also can contain or be attached to a navigational array 204. The surgeon, nurse, or other user touches the tip of probe 116 to a point such as a landmark on bone structure and actuates the foot pedal 110 or otherwise instructs the computer 108 to note the landmark position. The patient-mounted navigational sensor 100 “sees” the position and orientation of surgical reference 104 “knows” where the tip of probe 116 is relative to that surgical reference 104 and thus calculates and stores, and can display on monitor 114 whenever desired and in whatever form or fashion or color, the point or other position designated by probe 116 when the foot pedal 110 is hit or other command is given. Thus, probe 116 can be used to designate landmarks on bone structure in order to allow the computer 108 to store and track, relative to movement of the surgical reference 104, virtual or logical information such as mechanical axis 118, medial lateral axis 120 and anterior/posterior axis 122 of femur 102, tibia 101 and other body parts in addition to any other virtual or actual construct or reference.

A patient-mounted navigational sensor according to an embodiment of the present invention can communicate with suitable computer-aided surgical systems and processes such as the so-called FluoroNav system and software provided by Medtronic Sofamor Danek Technologies. Such systems or aspects of them are disclosed in U.S. Pat. Nos. 5,383,454; 5,871,445; 6,146,390; 6,165,81; 6,235,038 and 6,236,875, and related (under 35 U.S.C. Section 119 and/or 120) patents, which are all incorporated herein by this reference. Any other desired systems and processes can be used as mentioned above for imaging, storage of data, tracking of body parts and items and for other purposes.

The FluoroNav system can require the use of reference frame type fiducials which have four, and in some cases five elements, tracked by sensors for position/orientation of the fiducials and thus of the body part, implement, instrumentation, trial component, implant component, or other device or structure being tracked. Such systems can also use at least one probe 116 which the surgeon can use to select, designate, register, or otherwise make known to the system a point or points on the anatomy or other locations by placing the probe as appropriate and signaling or commanding the computer to note the location of, for instance, the tip of the probe. The FluoroNav system can also track position and orientation of a C-arm used to obtain fluoroscopic images of body parts to which fiducials have been attached for capturing and storage of fluoroscopic images keyed to position/orientation information as tracked by the sensors 100. Thus, the monitor 114 can render fluoroscopic images of bones in combination with computer generated images of virtual constructs and references together with implements, instrumentation components, trial components, implant components and other items used in connection with surgery for navigation, resection of bone, assessment and other purposes.

A patient-mounted navigational sensor according to various embodiments of the invention can be used with point of class-type, registration-type, and other surgical location and preparation techniques and methods. For example, in one prosthetic installation procedure, a surgeon can designate a center of rotation of a patient's femoral head for purposes of establishing the mechanical axis and other relevant constructs relating to the patient's femur according to which prosthetic components can ultimately be positioned. Such center of rotation can be established by articulating the femur within the acetabulum or a prosthesis to capture a number of samples of position and orientation information and thus in turn to allow the computer to calculate the average center of rotation. The center of rotation can be established by using a probe associated with a navigational array, and designating a number of points on the femoral head and thus allowing the computer to calculate the geometrical center or a center that corresponds to the geometry of points collected. Additionally, graphical representations such as controllably sized circles displayed on the monitor can be fitted by the surgeon to the shape of the femoral head on planar images using tactile input on screen to designate the centers according to that graphic, such as are represented by the computer as intersection of axes of the circles. Other techniques for determining, calculating or establishing points or constructs in space, whether or not corresponding to bone structure, can be used in accordance with the present invention.

In another example, a patient-mounted navigational sensor according to various embodiments of the invention can be used in designation or registration of items that will be used in surgery. Registration simply means ensuring that the computer knows which body part, item or construct corresponds to which fiducial or fiducials, and how the position and orientation of the body part, item or construct is related to the position and orientation of its corresponding fiducial or a fiducial attached to an impactor or other component which is in turn attached to an item. Such registration or designation can be done before or after registering bone or body parts. In one instance, a technician can designate with a probe an item such as an instrument component to which a navigational array is attached. A sensor associated with a computer-aided surgical navigational system can “see” the position and orientation of the navigational array attached to the item and also the position and orientation of the navigational array attached to the probe whose tip is touching a landmark on the item. The technician can designate onscreen or otherwise the identification of the item and then activates the foot pedal or otherwise instructs the computer to correlate the data corresponding to such identification, such as data needed to represent a particular cutting block component for a particular knee implant product, with the particularly shaped navigational array attached to the component. The computer has then stored identification, position and orientation information relating to the navigational array for the component correlated with the data such as configuration and shape data for the item so that upon registration, when the sensor can track the item and navigational array in the infrared field, the monitor can show the cutting block component moving and turning, and properly positioned and oriented relative to the body part or navigational information such as axes which is also being tracked.

Similarly, the mechanical axis and other axes or constructs of body parts can also be “registered” for tracking by the system. Again, the computer-aided surgical navigational system can employ a fluoroscope to obtain images of the patient's femoral head, knee and ankle, or other body parts, and/or it can allow generation of navigational information regarding such parts, such as for example, generation of mechanical axis information which can be displayed with the position and orientation of devices, components and other structures connected to navigational arrays. In the case of obtaining images, the system can correlate such fluoroscopic images with the position and orientation of the C-arm and the patient anatomy in real time as discussed above with the use of one or more navigational arrays placed on the body parts before image acquisition and which remain in position during the surgical procedure. Using these axes and constructs and/or images and/or the probe, the surgeon can select and register in the computer the center of the femoral head and ankle in orthogonal views, usually anterior/posterior and lateral, on a touch screen. The surgeon can use the probe to select any desired anatomical landmarks or references at the operative site of the knee or on the skin or surgical draping over the skin, as on the ankle. These points can be registered in three dimensional space by the system and can be tracked relative to the navigational arrays on the patient anatomy which are preferably placed intraoperatively. Although registering points using actual bone structure is one preferred way to establish the axis, a cloud of points approach by which the probe is used to designate multiple points on the surface of the bone structure can be employed, as can moving the body part and tracking movement to establish a center of rotation as discussed above. Once the center of rotation for the femoral head and the condylar component have been registered, the computer can calculate, store, and render, and otherwise use data for, the mechanical axis of the femur.

In one example, a tibial mechanical axis can be established by designating points to determine the centers of the proximal and distal ends of a patient's tibia so that the mechanical axis can be calculated, stored, and subsequently used by the computer. A posterior condylar axis can also determined by designating points or as otherwise desired, as rendered on the computer generated geometric images overlain or displayed in combination with the fluoroscopic images, all of which are keyed to one or more navigational arrays being tracked by sensors associated with the computer-aided surgical navigational system.

FIG. 2 illustrates a flowchart of a method 200 of use for a patient-mounted navigational sensor with a computer-aided surgical navigation system according to an embodiment of the invention.

The method 200 begins at block 202. At block 202, a navigational sensor is mounted to a body part of a patient. In the embodiment shown in FIG. 2, the navigational sensor can be similar to the patient-mounted navigational sensor 100 shown in FIG. 1. For example, a navigational sensor can include a sensor for sensing surgical references, and a mount adapted to be attached to the body part of a patient. In one embodiment, the sensor can be an optical tracking camera or infrared detector, for example, or any other sensor adapted to sense presence of an object on the navigational array. The navigational sensor in another embodiment can include at least two sensors for sensing surgical references and a mount adapted to be attached to the bone of a patient. In that embodiment, the at least two sensors may be for example, optical tracking cameras or infrared detectors, for example, or any other sensors adapted to sense presence of the surgical references.

Block 202 is followed by block 204, in which at least one surgical reference is mounted adjacent to an object. A mount associated with a navigational array, such as 104 shown in FIG. 1, can be utilized to support at least one surgical reference adjacent to an object, such as a body part of a patient. For example in this embodiment, an object can include at least one of the following: a bone, a tissue, a surgical implement, a surgical reference, a surgical trial, an implant, a cutting block, a reamer, a drill, a saw, an extramedullary rod, and an intramedullar rod.

Block 204 is followed by block 206, in which at least one surgical reference is sensed with the navigational sensor. As described above, the at least one surgical reference can be a navigational array 104 shown in FIG. 1. For example in one embodiment, the navigational sensor 100 can visually detect the presence of a passive-type surgical reference. In an example of another embodiment, the navigational sensor 100 can receive an active signal provided by an active-type surgical reference. A navigational sensor can sense, detect, or otherwise locate other suitable surgical references.

Block 206 is followed by block 208, in which a position associated with the object is determined based at least in part on sensing the surgical reference. As described above, associated computing functionality, such as 108 in FIG. 1, can process signals received from the navigational sensor to determine a position associated with the object. The computing functionality 108 can then correlate position and/or orientation information of surgical references with various types of images relative to relevant body part or parts, and facilitate display of the surgical references with respect to relevant body part or parts.

The method 200 ends at block 208. Other method elements can exist in accordance with embodiments of the invention.

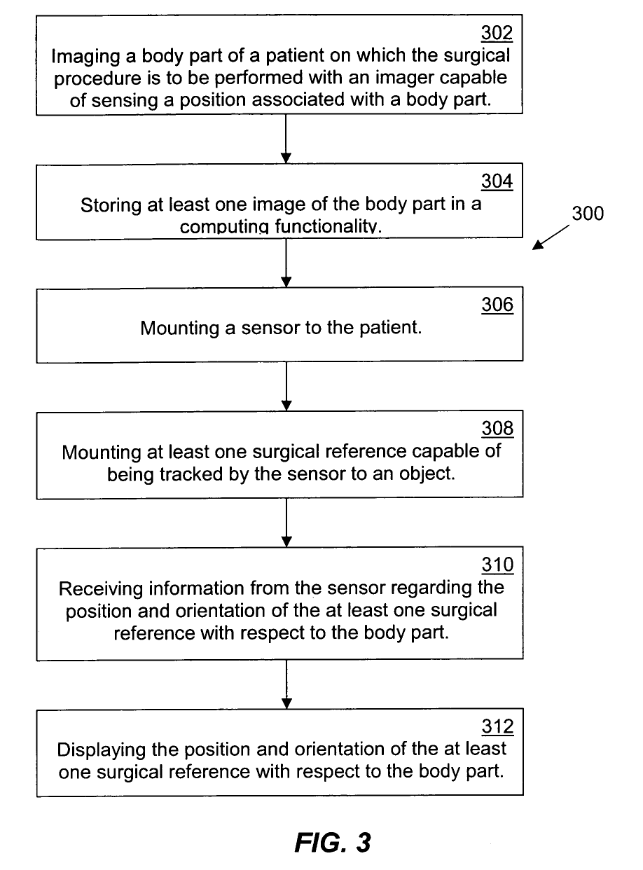

FIG. 3 illustrates a flowchart of a method of use for a computer-aided surgical navigation system with a patient-mounted navigational sensor according to an embodiment of the present invention.

The method 300 begins at block 302. At block 302, a body part of a patient on which the surgical procedure is to be performed is imaged. The imager can be an imager capable of sensing a position associated with the body part. As described above, the imager may be a C-arm that obtains fluoroscopic images of the desired body parts. The imager and the body parts can have a surgical reference attached to them so that a sensor “sees” and tracks the position of the imager as well as the positions and orientations of the body parts. An imager is not necessary; instead the system can instead generate and display relevant navigational information useful for correct orientation and placement of components and for navigation during surgery, such as mechanical axes, reference plane axes and/or other axes or navigational information mentioned at other places in this document. Block 302 is followed by block 304, in which at least one image of the body part is stored in a computing functionality, such as a computer, for example.

Block 304 is followed by 306, in which a sensor is mounted to the patient. The sensor is adapted to sense at least one surgical reference associated with an objection. The sensor is adapted to detect a position associated with at least one surgical reference. The sensor can be adapted to sense at least one of the following: an electric signal, a magnetic field, an electromagnetic field, a sound, a physical body, radio frequency, an x-ray, light an active signal or a passive signal. In some embodiments, the sensor may be a navigational sensor 100 as shown in FIG. 1, which includes two optical tracking cameras and a mount for associating the sensor to a body part of a patient.

Block 306 is followed by block 308, in which at least one surgical reference capable of being tracked by the sensor is mounted to an object. A surgical reference, such as 104 shown in FIG. 1 and described above, can be used. In some embodiments of the invention, the object is at least one of the following: a patient's bone, a patient's tissue, a patient's head, a surgical implement, a surgical reference, a surgical trial, an implant, a cutting block, a reamer, a drill, a saw, an extramedullary rod or an intramedullar rod.

Block 308 is followed by block 310, in which information is received from the sensor regarding the position and orientation of the at least one surgical reference with respect to the body part. As described above, associated computing functionality, such as 108 in FIG. 1, can process signals received from the sensor to determine a position associated with the object. The computing functionality 108 can then correlate position and/or orientation information of surgical references for display with various types of images, such as those received from the imager relative to the body part. Alternatively, the computing functionality 108 can correlate position and/or orientation information of surgical references for display with navigational information useful for correct orientation and placement of components and for navigation during surgery, such as mechanical axes, reference plane axes and/or other axes or navigational information mentioned at other places in this document. Alternatively, functionality 108 can correlate position and/or orientation of surgical references for display with a combination of such imaging and navigational information.

Block 310 is followed by block 312, in which the position and orientation of the at least one surgical reference with respect to the body part is displayed. Monitor 114, shown in FIG. 1 and described above, can be used to display the position and orientation of the at least one surgical reference with respect to the body part in combination with images of body parts or navigational information, or a combination of the two.

The above methods and techniques are provided by way of example only, and other embodiments of the present invention can be used with other surgical location and preparation techniques and methods.

Changes and modifications, additions and deletions may be made to the structures and methods recited above and shown in the drawings without departing from the scope or spirit of the invention and the following claims.

1. A computer-aided surgical navigation system comprising:
(a) a computer program adapted to generate navigational reference information regarding position and orientation of a patient's body part;
(b) a sensor mounted to the patient, the sensor adapted to track the position of at least one surgical reference;
(c) at least one surgical reference capable of being tracked by the sensor;
(d) a computer adapted to store at least some of the navigational reference information and to receive information from the sensor in order to track a position and orientation of the at least one surgical reference with respect to the body part; and
(e) a monitor adapted to receive information from the computer in order to display at least some of the navigational reference information and the at least one surgical reference.

2. The computer-aided surgical navigation system of claim 1, wherein the sensor is adapted to sense at least one of the following: an electrical signal, a magnetic field, an electromagnetic field, a sound, a physical body, radio frequency, an x-ray, light, an active signal, or a passive signal.

3. The computer-aided surgical navigation system of claim 1, wherein the sensor comprises:
(a) at least two optical tracking cameras for sensing at least one surgical reference associated with a body part of a patient, wherein the sensor is adapted to detect a position associated with the at least one surgical reference; and
(b) a mount adapted to be associated with the body part of the patient.

4. The computer-aided surgical navigation system of claim 1, wherein the body part is at least one of the following: a bone, a tissue, a patient's femur, a patient's head.

5. The computer-aided surgical navigation system of claim 1, further comprising an imager for obtaining an image of the body part of the patient, and wherein the computer is adapted to store the image in addition to the at least some of the navigational reference information, and the monitor is adapted to display the image and the at least some of the navigational reference information.

6. The computer-aided surgical navigation system of claim 1, wherein the navigational reference information includes at least one of a mechanical axis of a femur and a mechanical axis of the body part.

7. The computer-aided surgical navigation system of claim 1, wherein the navigational reference information relates to a bone on which the sensor is mounted.

8. The computer-aided surgical navigation system of claim 1, wherein the navigational reference information relates to a bone other than a bone on which the sensor is mounted.

9. A method for performing computer-assisted surgery using a patient-mounted navigational sensor comprising:
(a) mounting a navigational sensor to a body part of a patient, wherein the navigational sensor comprises:
(i) a sensor for sensing at least one surgical reference associated with a body part of a patient, wherein the sensor is adapted to detect a position associated with the at least one surgical reference; and
(ii) a mount adapted to be attached to the bone of a patient;
(b) mounting a surgical reference adjacent to an object;
(c) sensing the surgical reference with the navigational sensor; and
(d) determining a position associated with the object based in part on the sensing the surgical reference;
(e) in a computer, generating navigational reference information relative to said body part of the patient, wherein said navigational reference information includes at least one axis;
(f) displaying the position associate with the object based in part on the sensing of the surgical reference in combination with at least some of the navigational reference information.

10. The method of claim 9, wherein the object is a portion of the patient's body.

11. The method of claim 9, wherein the object is at least one of the following: a patient's bone, a patient's tissue, a patient's head, a surgical implement, a surgical reference, a surgical trial, an implant, a cutting block, a reamer, a drill, a saw, an extramedullary rod or an intramedullar rod.

12. The method of claim 9, wherein the sensor comprises at least two optical tracking cameras.

13. A method for performing a surgical procedure using a patient mounted navigational sensor and a computer-aided surgical navigation system, the method comprising:
(a) generating navigational reference information relating to position and orientation of a body part;
(b) storing at least some of said navigational reference information in a computer;
(c) mounting a sensor to the patient, wherein the sensor comprises:
(i) a sensor for sensing at least one surgical reference associated with an object, wherein the sensor is adapted to detect a position associated with the at least one surgical reference; and
(ii) a mount for associating the sensor to a body part of a patient;
(d) mounting at least one surgical reference capable of being tracked by the sensor to an object;
(e) receiving information from the sensor regarding the position and orientation of the at least one surgical reference with respect to the body part to which the sensor is mounted; and
(f) displaying the position and orientation of the at least one surgical reference with respect to the body part in combination with at least some of the navigational reference information.

14. The method of claim 13, wherein the sensor is adapted to sense at least one of the following: an electric signal, a magnetic field, an electromagnetic field, a sound, a physical body, radio frequency, an x-ray, light an active signal or a passive signal.

15. The method of claim 13, wherein the sensor comprises at least two optical tracking cameras for sensing at least one surgical reference associated with a body part of a patient.

16. The method of claim 13, wherein the body part is at least one of the following: a bone, a tissue, a patient's femur, a patient's head.

17. The method of claim 13, wherein the object is at least one of the following: a patient's bone, a patient's tissue, a patient's head, a surgical implement, a surgical reference, a surgical trial, an implant, a cutting block, a reamer, a drill, a saw, an extramedullary rod or an intramedullar rod.

18. The method of claim 13 further comprising:
(a) imaging the body part of the patient with an imager capable of sensing a position associated with a body part;
(b) storing at least one image of the body part in a computing functionality; and
(c) displaying the position and orientation of the at least one surgical reference with respect to the body part in combination with at least some of the navigational reference information and said at least one image.

19. The method of claim 13 wherein said navigational reference information includes at least one of a mechanical axis of a femur and a mechanical axis of the body part.

20. The method of claim 13 wherein said navigational reference information includes at least one of an axis corresponding to the anterior/posterior aspect and an axis corresponding to a medial/lateral aspect of the body part.

21. The method of claim 13 in which the sensor is a stereoscopic infrared sensor.
